(19) United States
(12) Patent Application Publication
(10) Pub. No.: US 2026/0042810 A1
(43) Pub. Date: Feb. 12, 2026

(54) A PROCESS FOR THE PREPARATION OF GLUCAGON

(71) Applicant: BIOCON LIMITED, Bangalore (IN)

(72) Inventors: Vaibhav Ramdas PAWAR, MAHARASHTRA (IN); Suprabha PRABHAKARA, BENGALURU (IN); Sandeep Rao S SRIPATHI, BENGALURU (IN); Nivrutti JOGDAND, PUNE (IN); Nitin Sopanrao PATIL, BENGALURU (IN)

(21) Appl. No.: 19/150,046

(22) PCT Filed: Jan. 25, 2024
(86) PCT No.: PCT/IB2024/050695

§ 371 Date: Jul. 22, 2025

(30) Foreign Application Priority Data
Jan. 27, 2023 (IN) 202341005417

(51) Int. Cl. C07K 14/605

(52) U.S. Cl. CPC C07K 14/605

(57) ABSTRACT

The present invention relates to a process for preparation of glucagon comprising condensing a fragment-1 (24 mer providing amino acid residues 6-29 of glucagon) with a fragment-2 (5-mer providing amino acid residues 1-5 of glucagon) in the presence of a coupling agent to obtain a protected glucagon, deprotecting the protected glucagon with a cocktail mixture to afford crude glucagon followed by RP-HPLC purification to isolate pure glucagon.

### RELATED APPLICATION

This application claims the benefit of priority of our Indian patent application IN 202341005417 filed on Jan. 27, 2023, which is incorporated herein by reference.

### FIELD OF THE INVENTION

The present invention relates to a process for the preparation of glucagon represented by Formula-I,

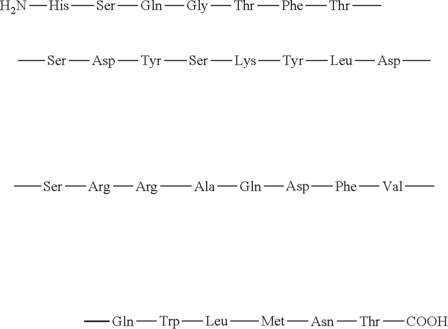

The present invention relates to a process for the preparation of glucagon by a solid phase peptide synthesis. Further, the present invention relates to a process for the preparation of glucagon by coupling fragments in a required sequence, deprotection and condensing them in solid phase, followed by RP-HPLC purification to get pure glucagon.

### BACKGROUND OF THE INVENTION

Glucagon is a linear peptide hormone consisting of 29 amino acid residues, secreted from the pancreatic α cells. Glucagon shares the same precursor, proglucagon, with GLP-1 and GLP-2. By tissue-specific posttranslational processing, glucagon is secreted from pancreatic α cells whereas GLP-1 and GLP-2 are secreted from intestinal L cells. All these peptides have considerable sequence homology and form the glucagon family, a subfamily of the secretin-glucagon superfamily.

Among the glucagon family members, the primary structure of glucagon is most highly conserved in vertebrates. Glucagon is the principal hyperglycemic hormone and acts as a counter balancing hormone to insulin. Glucagon generally elevates the blood glucose levels by promoting gluconeogenesis and glycogenolysis. Glucagon has the greatest effect on the liver, although it affects many different organs in the body such as the adipose tissue, pancreas, brain, and kidney.

U.S. Pat. No. 3,642,763 describes a synthesis of glucagon by condensation of [aa 1-6] and an [aa 7-29] peptide fragments in the presence of N-hydroxy-succinimide or N-hydroxypthalimide and subsequent splitting of protecting groups in the presence of trifluoroacetic acid.

Japanese Patent Application No. JP1995-146255 describes an Fmoc solid phase peptide synthesis of glucagon by sequential coupling of amino acids based on peptide backbone of glucagon.

PCT Application Publication No. 2020254479 describes a synthesis of glucagon by condensation of [aa 1-4] and an [aa 5-29] peptide fragments, C-terminal [aa 5-29] fragment consisting of atleast one pseduoproline dipeptide.

Chinese Patent No. CN103333239B describes an Fmoc solid phase peptide synthesis of Glucagon, wherein the coupling is performed at high temperature.

The inventors of the present invention used the existing synthesis method to prepare glucagon and found that the prior art has the technical problems of more synthetic steps, long synthesis cycle, low purity and yield, high production cost, and is not conducive to large-scale production. To this end, the inventors conducted research on the synthesis method of glucagon, thereby obtaining the technical solution of the present invention.

The present invention provides a process for the preparation of glucagon by coupling appropriate fragments in a required sequence, deprotection and condensing them in solid phase, followed by purification to get glucagon.

### SUMMARY OF THE INVENTION

A first embodiment of the invention relates to a process for preparing a glucagon as shown in scheme-1, the process comprising the steps of:
a. condensing a fragment-1 with a fragment-2 in the presence of coupling agent to obtain protected glucagon;
b. deprotecting the protected glucagon with a cocktail mixture to afford crude glucagon; and
c. purifying by RP-HPLC to isolate pure glucagon

In a first aspect of first embodiment, the process for the preparation of (fragment-1) comprises the steps of:
a) anchoring the first protected terminal amino acid to a Wang resin,
b) selectively deprotecting the amino group,
c) coupling carboxyl terminus of the next N-protected amino acid to the amine group, and
d) repeating steps b) and c) to form a peptide sequence [aa 6-29] of glucagon, wherein the peptide sequence attached to the resin.

In a second aspect of the first embodiment, the process for the preparation of (fragment-2) comprises the steps of:
a) anchoring the first protected terminal amino acid to a 2-chloro trityl chloride resin,
b) selectively deprotecting the amino group,
c) coupling carboxyl terminus of the next N-protected amino acid to the amine group,
d) repeating steps b) and c) to form a peptide sequence [aa 1-5] of glucagon, and
e) cleaving the fragment-2 from the resin.

A second embodiment provides the novel fragments 1, and 2 are as follows,

a)
H₂N-Phe-Thr(tBu)-Ser(tBu)-Asp(OtBu)-Tyr(tBu)-Ser(tBu)-Lys(Boc)-Tyr(tBu)-Leu-
Asp(OtBu)-Ser(tBu)-Arg(Pbf)-Arg(Pbf)-Ala-Gln(Trt)-Asp(OtBu)-Phe-Val-Gln(Trt)-
Trp(Boc)-Leu-Met-Asn(Trt)-Thr(tBu)-Wang resin [fragment-1]
b)
Boc-His(Trt)-Ser(tBu)-Gln(Trt)-Gly-Thr(tBu)-OH [fragment-2]

A third embodiment of the invention relates to a process for preparing a glucagon as shown in scheme-2, the process comprising the steps of:
a. condensing a fragment-3 with a fragment-4 in the presence of coupling agent to obtain protected glucagon;
b. deprotecting the protected glucagon with a cocktail mixture to afford crude glucagon; and
c. purifying by RP-HPLC to isolate pure glucagon

A fourth embodiment of the invention provides use of

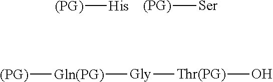

Wherein PG is a suitable amino protecting group.

In a first aspect of third embodiment, the process for the preparation of (fragment-3) comprises the steps of:
a) anchoring the first protected terminal amino acid to a Wang resin,
b) selectively deprotecting the amino group,
c) coupling carboxyl terminus of the next N-protected amino acid to the amine group,
d) repeating steps b) and c) to form a peptide sequence [aa 7-29] of glucagon, wherein the peptide sequence is attached to the resin

In a second aspect of third embodiment, the process for the preparation of (fragment-4) comprises the steps of:

a) anchoring the first protected terminal amino acid to a 2-chloro trityl chloride resin,
b) selectively deprotecting the amino group,
c) coupling carboxyl terminus of the next N-protected amino acid to the amine group,
d) repeating steps b) and c) to form a peptide sequence [aa 1-6] of glucagon, and
e) cleaving the fragment-2 from the resin.

A fourth embodiment provides the novel fragments 3, and 4 are as follows

a)
H₂N-Thr(tBu)-Ser(tBu)-Asp(OtBu)-Tyr(tBu)-Ser(tBu)-Lys(Boc)-Tyr(tBu)-Leu-
Asp(OtBu)-Ser(tbu)-Arg(Pbf)-Arg(Pbf)-Ala-Gln(Trt)-Asp(OtBu)-Phe-Val-Gln(Trt)-
Trp(Boc)-Leu_met-Asn(Trt)-Thr(tBu)-Wang resin [fragment-3]
b)
Boc-His(Trt)-Ser(tBu)-Gln(Trt)-Gly-Thr(tBu)-Phe-OH [fragment-4]

A fifth embodiment provides the purification of glucagon as follows,
a) subjecting the crude glucagon to a reversed phase high performance liquid chromatography (RP-HPLC purification) using an aqueous mobile phase comprising formic acid and acetonitrile/isopropanol mixture in a gradient elution manner;
b) collecting the fractions containing purified glucagon having HPLC Purity of greater than 98%

A sixth embodiment provides the purification of GLP-1 analogues selected from the group consisting of Liraglutide, Semaglutide, Glucagon, and Teduglutide comprising the step of RP-HPLC purification using a mobile phase comprising formic acid.

### DETAILED DESCRIPTION OF THE INVENTION

The best mode of carrying out the present invention is illustrated by the below mentioned examples. These examples are provided as illustration only and hence should not be construed as limitation to the scope of the invention.

### ABBREVIATIONS

Boc tert-butyloxycarbonyl
tBu tert-butyl
DCM dichloromethane
MDC Methylene dichloride
DIC or DIPC N,N′-diisopropylcarbodiimide
DMF N,N′-Dimethylformamide
DMAP Dimethyl amino pyrimidine
DIPEA or DIEA Diisopropylethylamine
HOBt Hydroxybenzotriazole
HBTU (2-(1H-benzotriazol-1-yl)-1,1,3,3-tetramethyluronium hexafluorophosphate
TFA Trifluoro acetic acid
MTBE tert-butyl methyl ether
HATU (1-[Bis(dimethylamino)methylene]-1H-1,2,3-triazolo[4,5-b]pyridinium 3-oxide hexafluorophosphate
TIS Triisopropyl silane
DTT Dithiotheritol
NH₄I Ammonium Iodide
Met L-Methionine
Trp L-Tryptophan
DIPC N,N′-Diisopropylcarbodiimide
DMAP 4-Dimethylaminopyridin
2-CTC resin 2-Chlorotrityl chloride resin
Fmoc 9-fluorenylmethoxycarbonyl
HOBt N-hydroxybenzotriazole
MTBE Methyl tert-butyl ether
His Histidine
Ser Serine
Gln Glutamine
Gly Glycine
Thr Threonine
Phe Phenylalanine
Asp Aspartic acid
Tyr Tyrosine
Lys Lysine
Leu Leucine
Arg Arginine
Ala Alanine
Gln Glutamine
Val Valine
Trp Tryptophan
Met Methionine
Asn Asparagine

The Fmoc deprotection of loaded amino acid according to the present invention is carried out using 0.05 to 0.5M Oxymapure in 5 to 15% piperidine in DMF or 0.05 to 0.5M formic acid in 5 to 15% piperidine in DMF, 0.05 to 0.5M HOBt in 5 to 15% piperidine in DMF or 1 to 5% DBU/0.1 to 1M Oxymapure in 5 to 15% piperidine in DMF or 1 to 5% DBU/0.05 to 0.5M HOBt in 5 to 15% piperidine in DMF. More preferably, 10% Piperidine in DMF is used.

Coupling of amino acids according to the present invention is carried out in the presence of coupling agent, coupling additive, inorganic salt and base. The coupling agent is selected from the group consisting of HBTU, HATU, COMU, DEPBT or DIC. Coupling additive was selected from the group consisting of Oxyma pure or HOBt, base was selected from DIPEA, NMM or TMP and inorganic salt selected from the group consisting of MgCl₂, CuCl₂ or ZnCl₂.

The process for the preparation of (fragment-1) comprises the steps of:
a) anchoring the first protected terminal amino acid to a Wang resin,
b) selectively deprotecting the amino group,
c) coupling carboxyl terminus of the next N-protected amino acid to the amine group,
d) repeating steps b) and c) to form a peptide sequence [aa 6-29] of glucagon, and
e) cleaving the fragment-1 from the resin.

### Example-1: Synthesis of Fragment-1 (24-Mer Providing Amino Acid Residues 6-29 of Glucagon)

### Stage-1: Swelling of the Wang Resin

Wang resin with 0.50 to 0.90 mmol/g substitution was taken in a reaction vessel and sufficient amount of DCM (10 to 15 Volumes) was added to it. The swelling procedure was allowed for 1-3 hrs at ambient temperature and drained.

Stage-2: Preparation of Fmoc-Thr (tBu)-Wang Resin

Required quantity of Fmoc-Thr (tBu)-OH (2.0 to 7.0 equivalents relative to the resin loading) was dissolved in DCM (3 to 6 Volumes). The amino acid solution was charged into the reaction vessel containing resin, followed by DIPC (3 to 9 equivalents) in DCM (3 to 5 Volumes) and DMAP (0.01 to 0.1 equivalents) in DCM (0.1 to 2 Volume). The reaction mass was kept under stirring for 1 to 3 hrs at ambient temperature. After the completion of coupling, repeated the washings with DCM and DMF three to five times each (5 to 15 Volumes).

### Stage-3: Capping

Capping was performed to block the unreactive sites. Solution of 5 to 10% acetic anhydride with 10 to 20% DIPEA in DMF (5 to 10 Volumes) was prepared and added to the reaction vessel containing first amino acid loaded resin. The reaction mass was stirred for 15 to 60 min at ambient temperature and drained. The process of capping was repeated for another 30 min and drained. Followed by four to six DMF washes (5 to 15 Volumes) were given.

### Stage-4: Fmoc-Deprotection

5 to 15% piperidine in DMF (5 to 15 Volumes) was added to the reaction vessel and stirred for 5 min at 15 to 30° C. and drained. Again, the resin was treated with 5 to 15% piperidine in DMF (5 to 15 Volumes) for 5 to 15 min at 15 to 30° C. and drained. Two HOBt·H₂O (0.1 to 1.0 M) in DMF (5 to 15 Volumes) washes were given. Two to five DMF washes (5 to 15 Volumes) were given. Kaiser test was performed to confirm the Fmoc deprotection before coupling the next amino acid.

After addition of Fmoc-Asp (OtBu)-OH to the sequence, continued the deprotection in 0.05 to 0.5M HOBt·H₂O in 5 to 15% piperidine in DMF (5 to 15 Volumes) and stirred for 5 to 15 min and drained. Again, the resin was treated with 0.05 to 0.5M HOBt in 5 to 15% piperidine in DMF (5 to 15 Volumes) for 5 to 15 min and drained. Two HOBt·H₂O (0.1 to 1.0 M) in DMF (5 to 15 Volumes) washes were given. Four DMF washes (5 to 15 Volumes) were given. Kaiser test was performed to confirm the Fmoc deprotection before coupling the next amino acid.

### Stage-5: Coupling of Fmoc-Amino Acids:

Weighed required amount of Fmoc-amino acid (1.0 to 5.0 equivalents), 1-hydroxy benzotriazole monohydrate (HOBt H₂O) (1 to 5 equivalents) and MgCl₂/CuCl₂/ZnCl₂ (0.5 to 1.0 equivalents). Dissolve them in 1 to 5 Volumes of DMF. Added the solution to peptide resin. Weighed required amount of HBTU/HATU (1.5 to 5.0 equivalents). Dissolve them in 1 to 5 Volumes of DMF. Added the solution to peptide resin, followed by addition of diisopropylethylamine (DIPEA) (3 to 8 equivalents). Stirred the mixture for about 5 hrs. Kaiser test was performed for the completion of the coupling reaction. Recoupling will be performed if test shows positive.

### Stage-6: Fmoc-Deprotection

0.05 to 0.5M HOBt·H₂O in 5 to 15% piperidine in DMF (5 to 15 Volumes) and stirred for 5 to 15 min at 20 to 40° C. and drained. Again the resin was treated with 0.05 to 0.5M HOBt·H₂O in 5 to 15% piperidine in DMF (10-20 Volumes) for 5 to 15 min at 20 to 40° C. and drained. HOBt·H₂O (2×0.1 to 1.0 M) in DMF (5 to 10 Volumes) washes were given. DMF washes (4×5 to 10 Volumes) were given. Kaiser test was performed to confirm the Fmoc deprotection before coupling the next 1-5 mer fragment.

The process for the preparation of (fragment-2) comprises the steps of:
a) anchoring the first protected terminal amino acid to a 2-chloro trityl chloride resin,
b) selectively deprotecting the amino group,
c) coupling carboxyl terminus of the next N-protected amino acid to the amine group,
d) repeating steps b) and c) to form a peptide sequence [aa 1-5] of glucagon, and
e) cleaving the fragment-2 from the resin.

In a preferred embodiment, the cleavage of fragment-2 from the resin occurs in a soft manner by using 0.5 to 5% TFA or 10 to 20% Trifluoroethanol. In the soft cleavage, the fragment-2 only cleaved from the attached resin and there is no side chain deprotection occurs in the fragment.

### Example-2: Synthesis of Fragment-2 (5-Mer Providing Amino Acid Residues 1-5 of Glucagon)

### Stage-1: Swelling of the CTC Resin

CTC resin with 1.0 to 1.5 mmol/g substitution was taken in reaction vessel and sufficient of amount of DCM (5 to 15 Volumes) was added to it. The swelling procedure was allowed for 1 to 3 h at 20 to 30° C. Drained the DCM using vacuum.

Stage-2: Preparation of Fmoc-Thr (tBu)-CTC Resin

Required quantity of Fmoc-Thr (tBu)-OH (1.0 to 4.0 equivalents relative to the resin loading) and required quantity DIPEA (3.0 to 8.0 equivalents relative to the resin loading) was dissolved in DCM (5 to 15 Volumes).

This solution was charged into the reaction vessel containing resin, the reaction mass was kept under stirring for 1 to 5 h at 20 to 35° C.

### Stage-3: Capping

Capping was performed to block the unreactive sites. Solution of 1- to 20% methanol with 1 to 20% DIPEA in DCM (5 to 20 Volumes) was prepared and added to reaction vessel containing first amino acid loaded resin. The reaction mass was stirred for 10 min to 60 min at 20 to 35° C. and drained. The process of capping was repeated for another 10 min to 60 min and drained. DMF washes (4×5 to 10 Volumes) were given.

After capping sample submitted for first loading estimation.

### Stage-4: Fmoc-Deprotection

5 to 20% piperidine in DMF (5 to 20 Volumes) was added to the reaction vessel and stirred for 5 min and drained. Again the resin was treated with 5 to 20% piperidine in DMF (5 to 20 Volumes) for 5 to 20 min and drained. Two HOBt·H₂O (0.1 to 1.0 M) in DMF (5 to 20 Volumes) washes were given. Four DMF washes (5 to 20 Volumes) were given. Kaiser test was performed to confirm the Fmoc deprotection before coupling the next amino acid.

### Stage-5: Coupling of Fmoc-Amino Acids:

Weighed required amount of Fmoc-amino acid (1 to 5 equivalents), 1-hydroxy benzotriazole monohydrate (HOBt H₂O) (1 to 5 equivalents) and MgCl₂, ZnCl₂ or CuCl₂ (0.5 equivalents). Dissolve them in 1 to 5 Volumes of DMF. Added the solution to peptide resin (after deblocking). Weighed required amount of HBTU/HATU (1 to 5 equivalents). Dissolve them in 1 to 5 Volumes of DMF. Added the solution to peptide resin, followed by addition of diisopropylethylamine (DIPEA) (3 to 8 equivalents). Stirred the mixture for 1.0-3.0 hrs. Kaiser test was performed for the completion of the coupling reaction. Recoupling will be performed if test shows positive.

### Stage-6a: Soft Cleavage Using Trifluoroacetic Acid

Prepared the 0.5 to 5% TFA in DCM (10 to 50 V), added 0.5 to 5% TFA in DCM (5 to 20 V) to the dried peptidyl resin. Stir it for about 5 min and drained. Collected the drain and neutralized it with DIPEA. Repeated the cycles of adding TFA to the peptidyl resin and draining for 1-5 times and collect all drains in one flask. The drain was concentrated to 10-40% with respect to the volume of cocktail mixture and concentrated by using rotary evaporator. Pre-chilled MTBE was slowly added to the concentrated mass under stirring. Solid precipitated was filtered and washed with MTBE.

### Or

### Stage-6b: Soft Cleavage Using Trifluoroethanol.

Prepared the 10 to 20% Trifluoroethanol (TFE) in DCM (10 to 50 V) and added to the dried peptidyl resin. Stir it for about 1.0 hr to 3.0 hr and drained. Collected the drain. The drain was concentrated to 10-40% with respect to the volume of cleavage mixture and concentrated by using rotary evaporator. Pre-chilled MTBE or hexane or heptane was slowly added to the concentrated mass under stirring. Solid precipitated was filtered and washed with MTBE or hexane or heptane.

A process for preparing a glucagon as shown in scheme-1, the process comprising the steps of:
a. condensing a fragment-1 with a fragment-2 in the presence of coupling agent to obtain protected glucagon;
b. deprotecting the protected glucagon with a cocktail mixture to afford crude glucagon; and
c. purifying by RP-HPLC to isolate pure glucagon

### Example-3: Synthesis of Glucagon

### Stage-1: Swelling of Fragment-1:

The fragment-1 (i.e., the 24-mer providing amino acid residues 6-29 of glucagon) on Wang resin obtained from Example-1 was swelled in 5-20 volumes of DMF, stirred for 1 to 3 hr and drained.

### Stage-2: Coupling of Fragment-2:

Weighed required amount of fragment-2, 3 equivalents (i.e. the 5-mer providing amino acid residues 1-5 of glucagon) obtained from Example-2, 1-hydroxy benzotriazole monohydrate (HOBt H₂O) (1 to 5 equivalents) and MgCl₂, ZnCl₂ CuCl₂ (0.5 to 3 0 equivalents). Dissolve them in 1 to 5 Volumes of DMF. Added the solution to peptide resin (after deblocking). Weighed required amount of HATU/HBTU (1-5 equivalents). Dissolve them in 1-5 Volumes of DMF. Added the solution to above peptide resin, followed by addition of diisopropylethylamine (DIPEA) (3 to 8 equivalents), stirred the mixture for 1-5 hrs. Kaiser test was performed for the completion of the coupling reaction. It should be negative if coupling reaction is over. Recoupling will be performed if test is positive.

### Stage-3: Total Cleavage

Charged 5 to 20 Volumes mixture of TFA: TIS: Phenol: DTT: NH4I: Met: Trp (81.5:5:5:2.5:2:2:2) respective to peptidyl resin to the well dried protected peptidyl resin. Stirred the reaction mixture for 1 to 6 hrs at room 10 to 40° C., filtered off the reaction mixture through a coarse filter funnel. The filtrate was concentrated to 20 to 60% with respect to the volume of cocktail mixture used by using rotary evaporator. Pre-chilled MTBE was slowly added to the concentrated mass under stirring. Solid precipitated was filtered and washed with MTBE to obtain crude glucagon.
Molar Yield: 90-95%
HPLC Purity: 50-60% Isolated crude glucagon was dried in VTD till achieved the consistent weight and proceed for RP-HPLC purification.

The process for the preparation of (fragment-3) comprises the steps of:
a) anchoring the first protected terminal amino acid to a Wang resin,
b) selectively deprotecting the amino group,
c) coupling carboxyl terminus of the next N-protected amino acid to the amine group,
d) repeating steps b) and c) to form a peptide sequence [aa 7-29] of glucagon, and
e) cleaving the fragment-3 from the resin.

### Example-4: Synthesis of Fragment-3 (23-Mer Providing Amino Acid Residues 7-29 of Glucagon)

### Stage-1: Swelling of the Wang Resin

Wang resin with 0.50 to 0.90 mmol/g substitution was taken in a reaction vessel and sufficient of amount of DCM (8 to 15 Volumes) was added to it. The swelling procedure was allowed for 1 to 4 hrs at 20 to 30° C. and drained.

Stage-2: Preparation of Fmoc-Thr (tBu)-Wang Resin

Required quantity of Fmoc-Thr (tBu)-OH (3 to 7.0 equivalents relative to the resin loading) was dissolved in DCM (2 to 5 Volumes). The amino acid solution was charged into the reaction vessel containing resin, followed by DIPC (5 to 10 equivalents) in DCM (2 to 5 Volumes) and DMAP (0.01 to 0.25 equivalents) in DCM (0.1 to 1.5 Volume). The reaction mass was kept under stirring for 1 to 5 hrs at 20 to 35° C. After completion of coupling DCM and DMF washes were given three times each (5 to 20 Volumes).

### Stage-3: Capping

Capping was performed to block the unreactive sites. Solution of 5 to 10% acetic anhydride with 10 to 25% DIPEA in DMF (5 to 10 Volumes) was prepared and added to reaction vessel containing first amino acid loaded resin. The reaction mass was stirred for about 30 min at 20 to 35° C. and drained. The process of capping was repeated for another 30 min and drained. DMF washes (4×5 to 20 Volumes) were given.

### Stage-4: Fmoc-Deprotection

5 to 20% piperidine in DMF (5 to 20 Volumes) was added to the reaction vessel and stirred for 5 to 10 min at 20 to 35° C. and drained. Again the resin was treated with 5 to 20% piperidine in DMF (5 to 20 Volumes) for 5 to 20 min at 20 to 35° C. and drained. Two HOBt·H₂O (0.1 to 1.0 M) in DMF (5 to 20 Volumes) washes were given. DMF washes (4×5 to 20 Volumes) were given. Kaiser test was performed to confirm the Fmoc deprotection before coupling the next amino acid.

After addition of Fmoc-ASP (OtBu)-OH to the sequence, continued the deprotection in 0.05 to 0.5 M HOBt in 5 to 20% piperidine in DMF (5 to 20 Volumes) and stirred for 5 to 10 min and drained. Again the resin was treated with 0.1 to 1 M HOBt in 5 to 20% piperidine in DMF (5 to 20 Volumes) for 5 to 20 min and drained. Two HOBt·H₂O (0.5 M) in DMF (5 to 20 Volumes) washes were given. DMF washes (4×5 to 20 Volumes) were given. Kaiser test was performed to confirm the Fmoc deprotection before coupling the next amino acid.

### Stage-5: Coupling of Fmoc-Amino Acids:

Weighed required amount of Fmoc-amino acid (2 to 5 equivalents), 1-hydroxy benzotriazole monohydrate (HOBt H₂O) (2 to 5 equivalents) and MgCl₂ or ZnCl₂ or CuCl₂ (0.1 to 1.0 equivalents). Dissolve them in 2 to 5 Volume of DMF. Added the solution to peptide resin (after deblocking). Weighed required amount of HBTU/HATU (2 to 5 equivalents). Dissolve them in 2 to 5 Volume of DMF. Added the solution to peptide resin, followed by addition of diisopropylethylamine (DIPEA) (2 to 9 equivalents). Stirred the mixture for 1.0-4 hrs. Kaiser test was performed for the completion of the coupling reaction. Recoupling will be performed if test shows positive.

### Stage-6: Fmoc-Deprotection

0.05 to 0.5 M HOBt·H₂O in 5 to 20% piperidine in DMF (5 to 20 Volumes) and stirred for about 5 min at 20 to 35° C. and drained. Again the resin was treated with 0.05 to 0.5 M HOBt·H₂Oin 5 to 20% piperidine in DMF (5 to 20 Volumes) for 5 to 20 min at 10 to 35° C. and drained. Two HOBt·H₂O (0.1 to 1.0 M) in DMF (5 to 10 Volumes) washes were given. Four DMF washes (8 Volumes) were given. Kaiser test was performed to confirm the Fmoc deprotection before coupling the next 1-5 mer fragment.

The process for the preparation of (fragment-4) comprises the steps of:
a) anchoring the first protected terminal amino acid to a 2-chloro trityl chloride resin,
b) selectively deprotecting the amino group,
c) coupling carboxyl terminus of the next N-protected amino acid to the amine group,
d) repeating steps b) and c) to form a peptide sequence [aa 1-6] of glucagon, and
e) cleaving the fragment-4 from the resin

In a preferred embodiment, the cleavage of fragment-4 from the resin occurs in a soft manner by using 0.5 to 5% TFA or 10 to 20% Trifluoroethanol. In the soft cleavage, the fragment-4 only cleaved from the attached resin and there is no side chain deprotection occurs in the fragment.

### Example-5: Synthesis of Fragment-4 (6-Mer Providing Amino Acid Residues 1-6 of Glucagon)

### Stage-1: Swelling of the CTC Resin

CTC resin with 1.0 to 1.5 mmol/g substitution was taken in reaction vessel and sufficient amount of DCM (5 to 20 Volumes) was added to it. The swelling procedure was allowed for 1 to 3 hr at 20 to 35° C. Drained the DCM using vacuum.

### Stage-2: Preparation of Fmoc-Phe-O-CTC Resin

Required quantity of Fmoc-Phe-OH (1.5 to 3.0 equivalents relative to the resin loading) and DIPEA (3 to 7 equivalents relative to the resin loading) was dissolved in DCM (5 to 20 Volumes). This solution was charged into the reaction vessel containing resin, the reaction mass was kept under stirring for 1 to 4 hrs at 20 to 35° C. The reaction mass was then drained, and the resin washed 3 with DMF (10 volume×3).

### Stage-3: Capping

Capping was performed to block the unreactive sites. Solution of 5 to 20% methanol with 2 to 7% DIPEA in DCM (5 to 20 Volumes) was prepared and added to reaction vessel containing first amino acid loaded resin. The reaction mass was stirred for about 30 min at 20 to 35° C. and drained. The process of capping was repeated and drained. DMF washes (4×10 Volumes) were given.

After capping, Fmoc-Phe-O-CTC Resin thus prepared was subjected to loading factor estimation which was found in the range of 1.2 mmol/gm to 1.6 mmol/gm.

### Stage-4: Fmoc-Deprotection

5 to 20% piperidine in DMF (5 to 20 Volumes) was added to the reaction vessel and stirred for about 5 min and drained. The resin was treated with 5 to 20% piperidine in DMF (5 to 20 Volumes) for 5 to 20 min and drained. HOBt·H₂O (2×0.05 to 0.50 M) in DMF (5 to 20 Volumes) washes were given. DMF washes (4×5 to 20 Volumes) were given. Kaiser test was performed to confirm the Fmoc deprotection before coupling the next amino acid.

### Stage-5: Coupling of Fmoc-Amino Acid

Weighed required amount of Fmoc-amino acid (1.5 to 5.0 equivalents), 1-hydroxy benzotriazole monohydrate (HOBt·H₂O) (2 to 5 equivalents) and MgCl₂ or ZnCl₂ or CuCl₂ (0.1 to 1.0 equivalent). Dissolve them in 2 to 5 Volumes of DMF. Added the solution to peptide resin (after deblocking). Weighed required amount of HBTU/HATU (2 to 5 equivalents). Dissolved them in 2 to 5 Volumes of DMF. Added the solution to peptide resin, followed by addition of diisopropylethylamine (DIPEA) (4 to 8 equivalents). Stirred the mixture for 1.0-4.0 hrs. Kaiser test was performed for the completion of the coupling reaction. Recoupling will be performed if test shows positive.

### Stage-6a: Soft Cleavage Using Trifluoroacetic Acid

Prepared the 0.5 to 2% TFA in DCM (30-60 Volumes). Added 0.5 to 2.0% TFA in DCM (5 to 20 Volumes) to the dried peptidyl resin, stirred it for about 2 min and drained. Collected the drain and neutralized it with DIPEA. Repeated the cycles of adding TFA to the peptidyl resin and draining for 3 to 5 times and collect all drains in one flask. The drain was concentrated to 10 to 40% with respect to the volume of cocktail mixture and concentrated by using rotary evaporator. Pre-chilled MTBE was slowly added to the concentrated mass under stirring. Solid precipitated was filtered and washed with MTBE.

### Or

Stage-6b: Soft Cleavage using Trifluoroethanol

Prepared the 10% to 20% Trifluoroethanol (TFE) in DCM (10 to 50 V) and added to the dried peptidyl resin. Stir it for about 1.0 hr to 3.0 hr and drained. Collected the drain. The drain was concentrated to 10-40% with respect to the volume of cleavage mixture and concentrated by using rotary evaporator. Pre-chilled MTBE or hexane or heptane was slowly added to the concentrated mass under stirring. Solid precipitated was filtered and washed with MTBE or hexane or heptane.

A process for preparing a glucagon as shown in scheme-1, the process comprising the steps of:
a. condensing a fragment-3 with a fragment-4 in the presence of coupling agent to obtain a protected glucagon;
b. deprotecting the protected glucagon with a cleavage mixture to afford crude glucagon; and
c. optionally purifying by RP-HPLC to isolate pure glucagon

### Example-6: Synthesis of Glucagon

### Stage-1: Swelling of Fragment-3

The fragment-3 (i.e., the 23-mer providing amino acid residues 7-29 of glucagon) on wang resin obtained from Example-4 was swelled in 10 to 20 Volumes of DMF, stirred for 1 to 3 hr and drained.

### Stage-2a: Coupling of Fragment-4

Weighed required amount of 1-6 mer fragment (2 to 5 equivalents), 1-hydroxy benzotriazole monohydrate (HOBt·H₂O) (2 to 5 equivalents) and MgCl₂ or ZnCl₂ or CuCl₂ (0.1 to 1.0 equivalent). Dissolve them in 3 to 6 Volumes of DMF. Added the solution to peptide resin (after deblocking). Weighed required amount of HATU (1.5 to 5.0 equivalents). Dissolve them in 3 to 6 Volumes of DMF. Added the solution to above peptide resin, followed by addition of diisopropylethylamine (DIPEA) (4 to 8 equivalents), stirred the mixture for 2-3 hrs. Kaiser test was performed for the completion of the coupling reaction. It should be negative if coupling reaction is over. Recoupling will be performed if test is positive.

### Or

### Stage-2b: Coupling of Fragment-4

Weighed required amount of 1-6 mer fragment (2 to 5 equivalents), 1-hydroxy benzotriazole monohydrate (HOBt·H₂O) (2 to 5 equivalents) and Diisopropyl carbodiimide (2 to 5 equivalents). Dissolve them in 3 to 6 Volumes of DMF. Added the solution to above peptide resin, stirred the mixture for 2-3 hrs. Kaiser test was performed for the completion of the coupling reaction. It should be negative if coupling reaction is over. Recoupling will be performed if test is positive.

### Stage-3: Total Cleavage

Charged 5 to 20 volumes mixture of TFA: TIS: Phenol: DTT: NH₄I: Met: Trp (81.5:5:5:2.5:2:2:2) respective to peptidyl resin to the well dried protected peptidyl resin. Stirred the reaction mixture for about 3 hr at room 15 to 35° C., filtered off the reaction mixture through a coarse filter funnel. The filtrate was concentrated to 20 to 50% with respect to the volume of cocktail mixture used by using rotary evaporator. Pre-chilled MTBE was slowly added to the concentrated mass under stirring. Solid precipitated was filtered and washed with MTBE to obtain crude glucagon.
Molar Yield: 90-95%
RP-HPLC Purity: 50-60%

Isolated crude glucagon was dried in vacuum and proceed for RP-HPLC purification.

1. A process for preparation of glucagon comprising:
a. condensing a fragment-1 (24 mer providing amino acid residues 6-29 of glucagon) with a fragment-2 (5-mer providing amino acid residues 1-5 of glucagon) in the presence of a coupling agent to obtain a protected glucagon;
b. deprotecting the protected glucagon with a cocktail mixture to afford crude glucagon; and
c. optionally purifying by RP-HPLC to isolate pure glucagon.

2. The process according to claim 1, wherein the coupling agent in step a) selected from the group consisting of HATU, DIPEA, HOBt and DIC.

3. The process according to claim 1, wherein step b) is performed with the cocktail mixture of TFA, TIS, Phenol, DTT, Met, Trp, and NH₄I.

4. The process according to claim 1, wherein the fragment-1 is

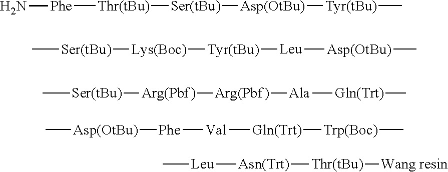

5. The process according to claim 1, wherein the fragment-2 is

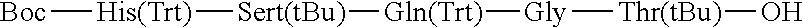

6. (canceled)

7. The process according to claim 1, further comprising the preparation of fragment-1, comprising:
a) anchoring the first protected terminal amino acid to a Wang resin,
b) selectively deprotecting the amino group,
c) coupling carboxyl terminus of the next N-protected amino acid to the amine group, and
d) repeating steps b) and c) to form a peptide sequence [aa 6-29] of glucagon,
wherein the peptide sequence attached to the resin.

8. The process according to claim 1, further comprising the preparation of fragment-2, comprising:
a) anchoring the first protected terminal amino acid to a 2-chlorotrityl chloride resin,
b) selectively deprotecting the amino group,
c) coupling carboxyl terminus of the next N-protected amino acid to the amine group,
d) repeating steps b) and c) to form a peptide sequence [aa 1-5] of glucagon, and
e) cleaving the fragment-2 from the resin.

9. A process for preparation of glucagon comprising:
a. condensing a fragment-3 (23 mer providing amino acid residues 7-29 of glucagon) with a fragment-4 (6-mer providing amino acid residues 1-6 of glucagon) in the presence of a coupling agent to obtain a protected glucagon;
b. deprotecting the protected glucagon with a cleavage mixture to afford crude glucagon; and
c. optionally purifying by RP-HPLC to isolate pure glucagon.

10. The process according to claim 9, wherein fragment-3 is

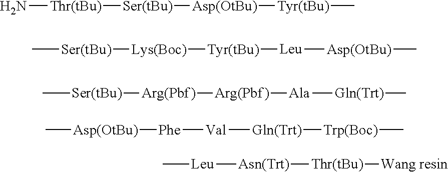

11. The process according to claim 9, wherein fragment-4 is,

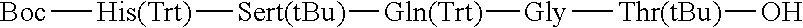

12. (canceled)

13. The process according to claim 9, further comprising the preparation of fragment-3, comprising:
a) anchoring the first protected terminal amino acid to a Wang resin,
b) selectively deprotecting the amino group,
c) coupling carboxyl terminus of the next N-protected amino acid to the amine group, and
d) repeating steps b) and c) to form a peptide sequence [aa 7-29] of glucagon,
wherein the peptide sequence attached to the resin.

14. The process according to claim 9, further comprising the preparation of fragment-4, comprising:
a) anchoring the first protected terminal amino acid to a 2-chlorotrityl chloride resin,
b) selectively deprotecting the amino group,
c) coupling carboxyl terminus of the next N-protected amino acid to the amine group,
d) repeating steps b) and c) to form a peptide sequence [aa 1-6] of glucagon, and
e) cleaving the fragment-4 from the resin.

15. The process according to claim 14, wherein fragment-4 is cleaved from the resin using Trifluoroethanol.
